(19) United States
(12) Patent Application Publication
(10) Pub. No.: US 2021/0047675 A1
(43) Pub. Date: Feb. 18, 2021

(54) METHODS FOR USING SPERMIOGRAMS TO SCORE VIABILITY OF SPERMATOZOA IN FRUIT FLY STERILE MALES

(71) Applicant: The United States of America, as Represented by the Secretary of Agriculture, Washington, DC (US)

(72) Inventor: Sandra N. LEAL, Harlingen, TX (US)

(21) Appl. No.: 16/991,529

(22) Filed: Aug. 12, 2020

Related U.S. Application Data
(60) Provisional application No. 62/885,424, filed on Aug. 12, 2019.

(51) Int. Cl. C12Q 1/24; G01N 1/28; C12Q 1/04

(52) U.S. Cl. CPC C12Q 1/24; G02B 21/34; C12Q 1/04; G01N 1/286

(57) ABSTRACT

A method for determining the relative viability of sterilized male fruit flies, focusing on examination of the male fruit fly teste or testes to enable consideration of the spermatozoa extruding from the vas deferens of the teste. The evaluation may determine relative viability of the spermatozoa; spermatogenesis; activity or motility of the spermatozoa; or morphological or physiological sperm cell abnormalities. The spermatozoa activity may also be captured in still or moving images, to allow further consideration based on the images.

### BACKGROUND

Fruit flies generally include two fly families—Tephritidae, and Drosophilidae (often called the “common fruit fly”). There are nearly 5,000 known species of tephritid fruit flies, in almost 500 genera of the Tephritidae family. See, for example, Wikipedia, at http://en.wikipedia.org/wiki/Tephritidae. Among the Tephritidae family, the genus Anastrepha is the most diverse genus in much of the Americas. This genus includes more than 300 known species, including the Mexican fruit fly, the South American fruit fly, the West Indian fruit fly, the sapote fruit fly, the Caribbean fruit fly, the American guava fruit fly, the pumpkin fruit fly, and the papaya fruit fly. See, for example, Wikipedia, at http://en.wikipedia.org/wiki/Anastrepha.

Fruit flies are often attracted to fresh and rotting fruit, as well as various parts of certain plants. Females deposit eggs in living, healthy plant tissue—including unripe fruit. Upon emerging from the eggs, the larvae feed on the ripening or rotting fruit, or various parts of the plants. Fruit flies can present a nuisance in the home or garden—affecting both growing plants and fruit, as well as ripe fruit brought into the house. See, Wikipedia, at https://en.wikipedia.org/wiki/Tephritidae.

However, agriculturally, fruit flies can present major ecologic and economic problems, causing significant damage to fruit and other plant crops. Some fruit flies feed on only one type of plant, while others are less specific. Crops that may be affected include olive plants, tropical fruit, vegetables, nut crops, celery and parsnips, sunflowers, and blueberries. See, Wikipedia, at https://en.wikipedia.org/wiki/Tephritidae.

As a result, there has been a lot of focus on controlling fruit fly infestations. Pest management techniques applied to tephritids have included use of conventional pesticides. However, due to the deleterious impact of pesticides, the trend has been to less impactful, more targeted methods. These methods include, for example, toxic food baits, male annihilation techniques using specific male attractant parapheromones in toxic baits or mass trapping. Other methods include sterile insect techniques. Id.

Sterile insect techniques are methods of biologically controlling insect populations. Typically, large numbers of sterile male insects are released in the desired geographic location. The sterile males compete with wild males to mate with wild females. Of course, any sterile male mating with a female will not produce offspring, thereby reducing the production of eggs. See Wikipedia, at https://en.wikipedia.org/wiki/Sterile_insect_technique. The goal of such sterile insect techniques is to have a large proportion of the females mate with sterile males, reducing the production of the next generation of insects.

Specifically for fruit flies, the Sterile Insect Technique (SIT) requires the release of the most competitive sterile males into the field, to decrease wild population levels. Mass reared flies undergo irradiation in order to achieve post-irradiation complete gonadal atrophy, resulting in sterility. The irradiation process affects the overall physiology of the insect.

Mass reared males should be able to efficiently transfer viable spermatozoa into the female sperm-storage organs via copulation. However, the female fruit fly is able to select the best quality sperm for storage, and to discriminate the stored sperm for purposes of fertilizing their eggs. Higher quality sperm is primarily stored in the Ventral Receptacle. The highest quality sperm is preferentially selected by the female fruit fly for fertilizing her eggs. Sperm competitiveness inside the female's sperm storage organs is an essential component for avoiding incidents of re-mating. The frequency of re-mating is determined by the quality and quantity of sperm stored in the female's sperm storage organs.

If a fertile inseminated female fruit fly receives irradiated, poor quality sperm from a non-competitive sterile male fruit fly, she will typically allow re-mating, with a non-irradiated, higher sperm quality male fruit fly; she may expel the irradiated, poor quality sperm previously stored. Similarly, if the female fruit fly tends to fail to mate with the mass-reared sterile male fruit fly, the efficacy of the SIT is greatly reduced.

In contrast, if the female fruit fly receives viable, fertile sperm, she will generally use the sperm to fertilize her eggs. Again, this undermines and reduces the desired effect of the SIT program.

Current operational protocols generally do not focus on determining the efficacy of the sterile male fruit flies released to mate with wild female fruit flies. A determination of the efficacy of a particular sterile male fruit fly population will enable program management to determine whether to try a different group of sterile male fruit flies, or even a different method of sterilizing the male fruit flies.

An accurate, reliable method of determining the effectiveness of SIT sterile male fruit flies on their mating patterns with female fruit flies, and thus the prevention of further fruit fly propagation, would provide important information useful in controlling populations of flies, enabling better control of economic and ecological effects, and of the measures taken to try to alleviate those deleterious effects.

### SUMMARY

We have developed a novel technique for conducting a spermiogram to analyze fruit fly semen, for determining the effectiveness of sterile male flies that are intended to be used in a SIT. This new technique includes the micro-dissection of the male fruit fly gonads (testes), and in vivo visualization of sperm movement at the end of the vas deferens. This new technique provides a reliable quality assurance/quality control tool for predicting the value and effectiveness of different sterile fruit fly strains intended to be released in the field. The sperm analyses, or spermiograms, enable in vivo or in vitro evaluation of the concentration, structure, and activity of the spermatozoa directly from the male's testes. The “in vivo” technique is a live spermiogram, analyzing live specimens from QA/QC mass colonies or SIT-released flies. The “in-vitro” spermiogram measures different histological findings to include spermiogenesis and assessment of sperm quality from the flies trapped in the field—dead specimens.

Our process determines the sterile (irradiated) vs. fertile (wild) status of the trapped male fruit flies based on the histological evaluation of the male's testes. This analysis provides a valuable added quality control test, evaluating the sterile male flies in order to help determine the laboratory colonies performance in real-time.

The spermiogram process may be used to analyze qualitative and/or quantitative factors regarding the viability of spermatozoa, as well as appropriate spermatogenesis, aiding the early detection of morphological/physiological sperm cell abnormalities within the testes.

The results of this analysis can help guide the potential recommendation of timely corrective measures intended to ensure the release of viable, competitive sterile insects into the fields. This can be used to help maximize the chances of the SIT program resulting in the desired effect on the wild population.

Accordingly, in one embodiment the present subject matter relates to a method of determining the viability of sterile male fruit fly spermatozoa, comprising:

(a) obtaining a sterile male fruit fly from a particular sterilized population,

(b) isolating from the sterile male fruit fly at least one fruit fly teste together with its respective vas deferens, and

(b) examining the condition of spermatozoa extruding from the vas deferens.

### BRIEF DESCRIPTION OF THE DRAWINGS

FIG. 1 shows a male fly in a petri dish with sterile saline solution or Ringer's Solution, ready for visualization under a dissecting microscope.

FIG. 2 shows a fly grasped by the thorax using angled forceps, with Vannas scissors being used to make an incision through the wall of the abdomen along the dorsal midline, from the apex to the base.

FIG. 3 shows the fly, with reproductive organs exposed, on a microscope slide, ready for dissection of the testes.

FIG. 4 shows the magnified vas deferens, and the free spermatozoa extruding from the vas deferens.

### DETAILED DESCRIPTION OF THE PREFERRED EMBODIMENTS

The present method provides a technique for determining the effectiveness of sterile male fruit flies that are intended to be used in a SIT program, to mate with wild female fruit flies without resulting in fertilized eggs and viable offspring. This new technique uses spermiogram analysis of sterile male fruit flies to determine their viability, and the attractiveness of the sterile male fruit fly spermatozoa to wild female fruit flies. This use of spermiogram analysis provides a means to reliably determine quality assurance and quality control regarding the sterile male fruit flies to be released for mating with wild female fruit flies, in an attempt to control the wild fruit fly population.

The method involves micro-dissection of the male gonads (testes) of sterile male fruit flies, and in vivo visualization of sperm movement at the end of the vas deferens from the sterile male fruit flies. The fruit fly spermiogram is carried out undiluted, also known as a wet technique. The cover slipped area is used to determine the volume required to allow accurate measurements of the concentration of spermatozoa, under microscopic examination.

This evaluation enables consideration of the concentration, structure, and activity of the spermatozoa, to help determine the laboratory colonies' performance in real-time.

The process analyzes the viability of sterile male fruit fly spermatozoa, as well as appropriate spermatogenesis, aiding the early detection of morphological/physiological sperm cell abnormalities within the testes. The results of this analysis can help suggest and guide the use of timely corrective measures to help ensure the release of viable, competitive sterile insects into the field, improving the prospects of the SIT program successfully resulting in the reduction of the wild population.

This process helps fill a gap in the current methodology. Typical SIT programs do not account in this manner for the ability of the physiology of wild female fruit flies to discern between viable sperm and sperm that is not viable, and potentially to eject sperm from sterile male fruit flies in favor of wild male spermatozoa. This analysis of the viability of sterile male fruit fly spermatozoa will help strengthen the field practitioner's ability to reliably prepare and release effective sterile male fruit flies into the subject population.

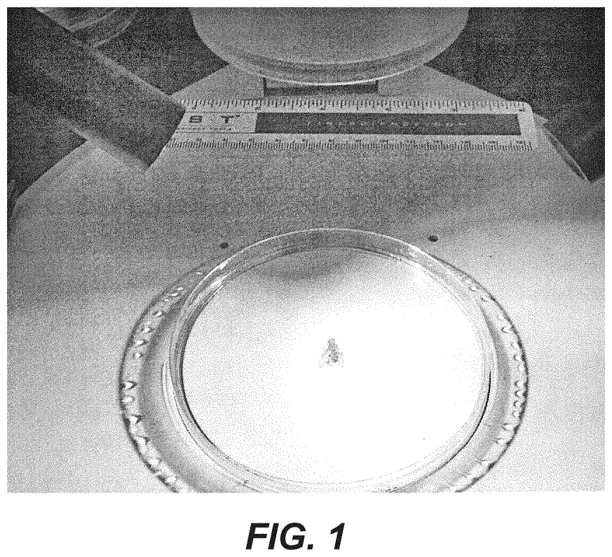

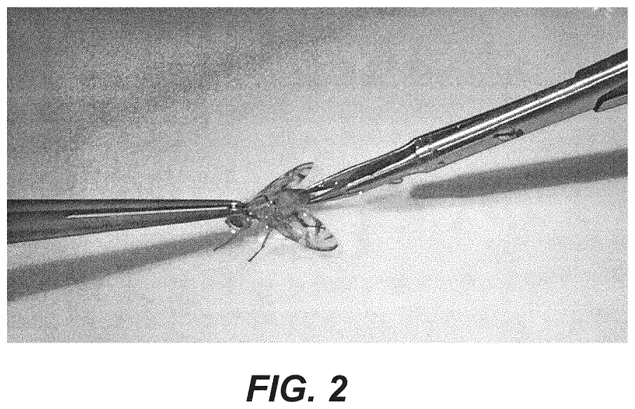

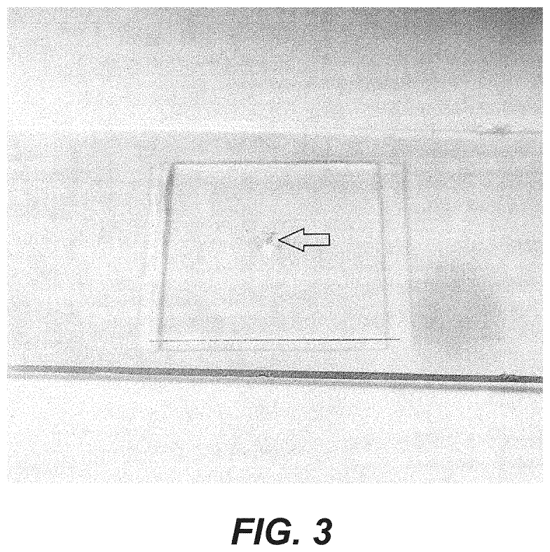

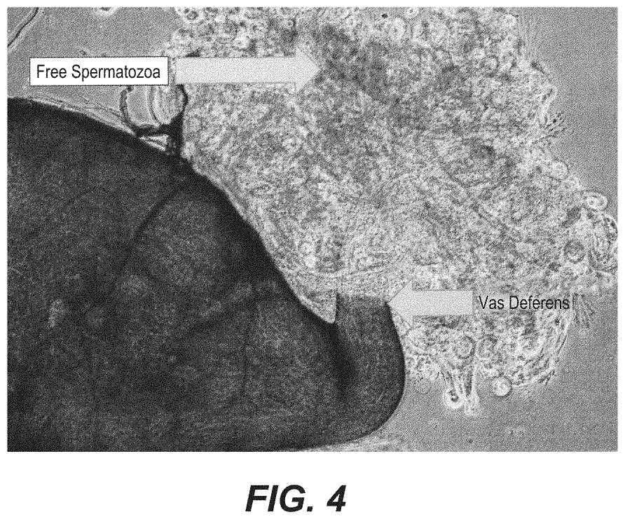

One embodiment of the present subject matter is summarized in FIGS. 1-4. A fruit fly to be examined is placed in a petri dish with sterile saline solution or Ringer's Solution. This enables visualization under the dissecting microscope during the procedure. FIG. 1. The fly's abdomen is cut open along the dorsal midline from the apex to the base. FIG. 2. This exposes the reproductive organs of the fly. Next, the testes are separated from the fly's body, by using forceps to grasp the testes at a point near the vas deferens, and gently pulling the testes away from the fly's abdomen. Each teste is placed on a microscope slide, with a drop of Ringer's solution or sterile water, and gently covered with a slide cover-slip. See FIG. 3. The specimen can then be examined, such as by using a compound microscope. In order to evaluate the viability and movement of spermatozoa, the specimen may be examined under successively increasing magnification up to 1000×, using an oil immersion lens. FIG. 4 shows free spermatozoa extruding from the vas deferens. If possible, recording an in vivo video provides active record of the spermatozoa activity and motility.

One embodiment of the present subject matter is a method for determining the viability of sterile male fruit fly spermatozoa. The method involves isolating the testes of the male fruit fly, and examination under magnification to determine activity and motility.

Another embodiment of the present subject matter is a method for determining the viability of sterile male fruit fly spermatozoa, and considering appropriate spermatogenesis, to help detect morphological and physiological sperm cell abnormalities. The method involves use of a dissecting microscope to enable dissection of the testes from the fly and use of a compound microscope to examine the spermatozoa exuding from the vas deferens, and optionally the use of a digital camera to capture still or active images of the spermatozoan activity.

Another embodiment of the present subject matter is a method for determining the viability of sterile male fruit fly spermatozoa. The method involves recordation of in vivo video of the spermatozoa activity, to document the sperm motility and activity.

The sperm analyses, or spermiograms, enable in vivo or in vitro evaluation of the concentration, structure, and activity of the spermatozoa directly from the male's testes.

A spermiogram is the most reliable test to directly measure, in real time, male fertility. The spermiograms provide real-time results that allow the detection of abnormalities, the determination of the presence or absence of pathogens within the testes, including for example parasites, bacteria, or protozoans, and provide reliable, timely remediation recommendations that will serve as scientific evidence to assist upper management with a better-informed decision-making process. This analysis provides a valuable added quality control test, evaluating the sterile male flies in order to help determine the laboratory colonies performance in real-time.

The process may be used to analyze qualitative and/or quantitative factors regarding the viability of spermatozoa, as well as appropriate spermatogenesis, aiding the early detection of morphological/physiological sperm cell abnormalities within the testes. Qualitative factors may include, for example, sperm morphology, physiology, and motility. Quantitative factors may include, for example, total spermatozoa count, sperm volume, and spermatozoa/seminal fluid volume ratio.

The results of this analysis can help guide the potential recommendation of timely corrective measures intended to ensure the release of viable, competitive sterile insects into the field. This can be used to help maximize the chances of the SIT program resulting in the desired effect on the wild population.

Normal results of a spermiogram test, or “normospermia,” indicate normal spermatogenesis, including normal quantity, morphology, and physiology of the spermatozoa. Male flies with these normal QA/QC values are the most effective in the field. Such male flies will generally transfer adequate volumes of sperm and seminal fluids into the female recipients. This will avoid female re-mating and result in effectiveness of the Sterile Insect Techniques by achieving suppression of the wild populations in the field.

Assessments conducted during a spermiogram can include sperm motility; sperm morphology; and concentration of volume, seminal fluid:spermatozoa ratio.

### Assessment of Sperm Motility

Category A: Normal or Typical motility is indicated by active movement and linear/circular displacement of the fruit fly spermatozoa.
Category B: Abnormal or Atypical motility is indicated by the non-progressive movement from the flagellum to the linear head of the fruit fly spermatozoa.
Category C: Immotility is indicated by the absence of spermatozoa movement.

### Assessment of Morphology

Category 1. Normal or Typical linear head and normal flagellum demonstrated by the fruit fly spermatozoa.
Category 2. Abnormal or Atypical head (curved, loop) or flagellum demonstrated by the fruit fly spermatozoa.

### Concentration of Volume, Seminal Fluid/Spermatozoa Ratio

This parameter measures the fruit fly's testicular capacity to produce sperm. The total sperm count in the ejaculate is multiplied by the seminal fluid volume, if we need to be exact. For practicality and daily practice, the volume of seminal fluid should not exceed the total number of spermatozoa in the cover slipped specimen. This visual assessment is based on training and daily practice.

If the sterile male's spermatozoa are of perceptively lower quality, or abnormal, the fertile females are likely to re-mate with another male—often a wild mate rather than another sterile male. Re-mating with a wild mate displaces the inferior sperm and replaces it with good quality fertile sperm from wild mates. This, of course, tends to defeat the purpose of releasing sterile males in a particular area, and to result in the establishment and growth of wild populations and larvae-infested areas.

Abnormal results of a spermiogram test can indicate one or more of at least the following possibilities:
Oligospermia: abnormally low sperm cell count. For male flies prepared to be infertile, this could result from issues such as abnormal or inadequate irradiation processes as one of the most common causes, but this finding just opens the door to many other possible causal factors.
Atypical sperm morphology: For male flies prepared to be infertile, this could result from issues such as improper irradiation, genotypic mutation, or genetic recombinants in the mass colony.
Atypical decreased sperm motility: The most probable cause in male flies prepared to be infertile would be improper irradiation, but it could also indicate genetic recombinants or substandard quality due to entomopathogens.
Decreased seminal fluid: This is most likely due to entomopathogens.

### The Examples

### Materials and Methods

We used the following materials and methods in practicing our new method.

Equipment

### Basic Microscopy System

a) Dissecting stereo-microscope, magnification required from 0.8× to 50×.
b) Compound microscope, magnification from 40× to 1000× (using immersion oil).
c) Digital camera for capturing the images.

### Micro-Dissection Tools

a) Micro-dissection scissors, Vannas style, cutting edge 2 mm.
b) Micro-dissection forceps, straight tip, 0.025×0.005 mm.
c) Micro-dissection forceps, 45-degree angle, serrated tip.

### Microscope Slides

a) Adhesion super frost slides, 25×75×0.1 mm.
b) Cover glass 18×18 mm.

### Immersion Oil

a) Low viscosity immersion oil for 1000× microscopy.

### Methodology

### Example 1—Determination of Spermatozoa Viability in Sterile Male Flies

1. Place the fly in a petri dish with sterile saline solution or Ringer's Solution. Visualize under the dissecting microscope. FIG. 1.
2. Grasp the fly by the thorax using the angled forceps. Use the Vannas scissors to cut open the abdomen, making an incision along the dorsal midline through the wall of the abdomen from the apex to the base. FIG. 2. This exposes the reproductive organs of the fly.
3. Dissect the testes by using the straight-tip forceps to grasp the testes at a point near the vas deferens, and gently pulling the testes away from the abdomen. Place each teste on the microscope slide, apply a drop of Ringer's solution or sterile water, and gently cover with a slide cover-slip. See FIG. 3.
4. Examine the specimen on the slide using the compound microscope objective at 20× magnification. To evaluate the viability and movement of spermatozoa, view the specimen under successively increasing magnification up to 1000× (oil immersion lens) and obtain in vivo video if possible. FIG. 4 shows free spermatozoa extruding from the vas deferens. The specimen may be viewed under successively increasing magnification, up to 1000× (using an oil immersion lens). Any specific magnification within the range of 40× to 1000× is contemplated as within the scope of the present subject matter, including but not limited to 50×, 60×, 70×, 80×, 90×, 100×, 200×, 300×, 400×, 500×, 600×, 700×, 800×, and 900×. Further, any contemplated range of magnifications can use any of the above as endpoints of the range. Thus, the specimen can be viewed at one magnification level, or at multiple magnification levels. FIG. 10 shows a magnified, ruptured ventral receptacle with spermatozoa having spilled from the ruptured alveoli. A live view (rather than a still photograph) may also enable evaluation of the degree of activity of any spermatozoa that are present.

It is to be understood that the new method described here is not limited to the specific embodiments described above, but instead encompasses any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A method of determining viability of sterile male fruit fly spermatozoa, comprising:
(a) obtaining a sterile male fruit fly from a particular sterilized population,
(b) isolating from the sterile male fruit fly at least one fruit fly teste together with its respective vas deferens, and
(c) examining the condition of spermatozoa extruding from the vas deferens.

2. The method of claim 1, wherein the examining is conducted under magnification.

3. The method of claim 2, wherein the magnification is provided by use of a microscope.

4. The method of claim 1, wherein the teste and vas deferens are placed on a microscope slide for examination using a microscope.

5. The method of claim 4, wherein the examination considers the spermatozoa's activity and motility.

6. The method of claim 4, wherein the examination is conducted to analyze spermatogenesis of the spermatozoa.

7. The method of claim 5, wherein the examination includes capturing still or moving images of the spermatozoan activity.

8. The method of claim 6, wherein the examination includes capturing still or moving images of spermatozoan activity.

9. A method of determining viability of sterile male fruit fly spermatozoa, comprising
(a) isolating at least one male fruit fly teste together with its respective vas deferens, and
(b) examining spermatozoa extruding from the vas deferens to detect morphological or physiological sperm cell abnormalities.

10. The method of claim 9, wherein the examination also determines relative activity and motility of the spermatozoa extruding from the vas deferens.

11. The method of claim 9, wherein the examination evaluates spermatogenesis.

12. The method of claim 11, wherein the examination evaluates a level of morphological or physiological sperm cell abnormalities.

13. The method of claim 10, wherein the examination includes capturing still or moving images of the spermatozoan activity to permit further analysis of the activity.

14. The method of claim 11, wherein the examination includes capturing still or moving images of spermatozoan activity.

15. The method of claim 12, wherein the examination includes capturing still or moving images of spermatozoan activity.
